(19) United States
(12) Patent Application Publication
(10) Pub. No.: US 2006/0285696 A1
(43) Pub. Date: Dec. 21, 2006

(54) High Noise Environment Stethoscope

(76) Inventor: Andrianus J.M. Houtsma, Enterprise, AL (US)

Correspondence Address: OFFICE OF THE STAFF JUDGE ADVOCATE;U.S. ARMY MEDICAL RESEARCH AND MATERIEL COMMAND, ATTN: MCMR-JA (MS. ELIZABETH ARWINE), 504 SCOTT STREET, FORT DETRICK, MD, 21702-5012, US

(21) Appl. No.: 11/425,312

(22) Filed: Jun. 20, 2006

Related U.S. Application Data
(60) Provisional application No. 60/692,257, filed on Jun. 21, 2005.

(51) Int. Cl. A61B 7/04

(52) U.S. Cl. 381/67

(57) ABSTRACT

A stethoscope having noise suppression features that make it suitable for use in high noise environments. The stethoscope includes ear pieces connected to signal processing components connected to a transducer in a chest piece that includes at least one suppression barrier extending from it. In at least one embodiment, the transducer is centered on the chest piece with the at least one suppression barrier centered around the transducer. The suppression barrier prevents surface waves resulting from environmental noise from reaching the transducer thus providing a higher quality signal to be processed and in turn listened to by the medical professional.

### I. FIELD OF THE INVENTION

This invention relates to a new and improved stethoscope that is capable of being used in high noise environments such as on medical evacuation flights and sporting events.

### II. BACKGROUND OF THE INVENTION

The classical stethoscope is an acoustic device that is used to pick up sounds originating from moving tissue inside the body at the surface of a patient's skin, and to transmit these sounds to the ears of a listener, for example an examining medical professional. A rim around the stethoscope membrane (or diaphragm) of the classical stethoscope forms an air pocket at the observation site so that the resulting air pressure will act as the transmission medium for pressure waves formed by movement of the skin to travel to the stethoscope membrane present in a chest piece for further transmission to the listener via tubing, a binaural, and ear tips. This process is commonly referred to as auscultation.

To assist medical professionals in performing auscultation, electronic stethoscopes have been developed that use a mechanical-electrical vibration member, e.g., a transducer, present in a chest piece that is placed against the patient's skin to directly receive vibrations caused by the heart beating and the individual breathing on the skin of the patient. One reason for electronic stethoscopes is that the user is able to enhance the sounds to better provide a diagnosis. Typically the user is able to adjust the volume or the frequency response to increase the range of sounds able to be listened to by the user. The sound enhancement is typically handled by signal processing components that may be located within the chest piece or at some other point in the transmission path between the transducer and the ear pieces.

There is however a problem that exists with stethoscopes of any type. In noisy environments it is often difficult if not impossible to hear any body sounds from the patient over the environmental noise. Some examples of high noise environments where auscultation of patients may be performed are battlefield or medical evacuation flights in helicopters, sporting events, accident scenes, construction sites, etc. These environments make it very difficult or impossible to perform auscultation because of high background noise levels, which can be as high as 95 to 120+ dBA.

In the case of medical evacuations, for instance, helicopter noise invades the stethoscope system through the patient's body from Surface Acoustic(al) Waves or SAW waves created in the patient's skin from the environmental noise. The SAW waves cause additional movement of the transducer, which results in signal noise being created and processed by the electronic stethoscope for delivery to the user. So even if the medic is wearing ear protection such as integrated headphones or earmuffs over the ear pieces to block noise from the helicopter, the SAW waves make it difficult if not impossible to use auscultation until the noise decreases. This phenomenon will also occur with a diaphragm or bell arrangement on non-electrical stethoscopes.

Another example of a high noise environment is sporting events—in both indoor and outdoor venues—where the noise level at different points may reach upwards of 120 dBA. In these environments, it is critical for medical professionals to be able to quickly detect heart and lung activity. However, the high noise levels would make it impossible to tend to an injured person, particularly a fan attending the event or a player in the bench area, and detect any heart or breathing sounds.

Environmental noises tend to enter the stethoscope through all components of the stethoscope system including the ear/stethoscope interface, tubing and sensor head. There have been many improvements to these components directed towards isolating the sound that the medical provider is interested in, i.e. heart and lung activity. Through the use of various components and techniques, such as integrated headsets or earmuffs; shielding the tubing or replacing the tubing with wires; acoustical noise reduction, shielding or impedance matching on the sensor head; and/or filtering the resulting signal, much of the environmental noises that enter the system can be blocked or filtered. These components and techniques allow a medical provider to listen to clearer signals, free of extraneous noise.

However, noise is still capable of entering the systems most commonly at the sensor head, as the sensor head of existing stethoscopes remains largely unprotected. Therefore, notwithstanding the usefulness of the above-described existing stethoscopes, a need still exists for a stethoscope for use in high noise environments.

### III. SUMMARY OF THE INVENTION

This invention provides an improved stethoscope having a barrier that suppresses Surface Acoustic(al) Waves traveling through a patient's skin parallel to the dermal layers.

An objective of at least one embodiment of the invention is to prevent noise-excited SAW waves from reaching the transducer/diaphragm of the stethoscope.

An advantage of at least one embodiment of the invention is improved ability of the user to hear heart and lung activity of the patient in high noise environments.

In an exemplary embodiment the present invention discloses a stethoscope including: a pair of ear pieces; signal processing components connected to said ear pieces; and a chest piece having a housing a transducer connected to said signal processing components and said housing, at least one suppression barrier attached to said housing.

In another exemplary embodiment the present invention discloses a chest piece for use in an electronic stethoscope including: a housing; signal processing components within said housing, said signal processing components includes a connection interface; a transducer connected to said signal processing components; and at least one suppression barrier attached to said housing and centered with respect to said transducer.

In still another exemplary embodiment the present invention discloses a stethoscope including: ear pieces; a binaural connected to said ear pieces; tubing connected to said binaural; a chest piece connected to said tubing, said chest piece having a housing attached to said tubing, a diaphragm or bell connected to said housing, a rim around said diaphragm or bell; and at least one suppression barrier attached to said housing and extending from said housing external to said rim.

Given the following enabling description of the drawings, the apparatus should become evident to a person of ordinary skill in the art.

### IV. BRIEF DESCRIPTION OF THE DRAWINGS

The present invention is described with reference to the accompanying drawings. In the drawings, like reference numbers indicate identical or functionally similar elements. The use of cross-hatching and shading within the drawings is not intended as limiting the type of materials that may be used to manufacture the inventions but is used to illustrate the output of light and its reflection, if any.

FIG. 1 illustrates a sketch of an exemplary transducer chest piece schematic according to the invention.

FIG. 2 depicts an exemplary transducer chest piece according to the invention.

FIG. 3 illustrates another exemplary transducer chest piece according to the invention.

FIG. 4 depicts a cross-section of an exemplary acoustic chest piece according to the invention.

### V. DETAILED DESCRIPTION OF THE DRAWINGS

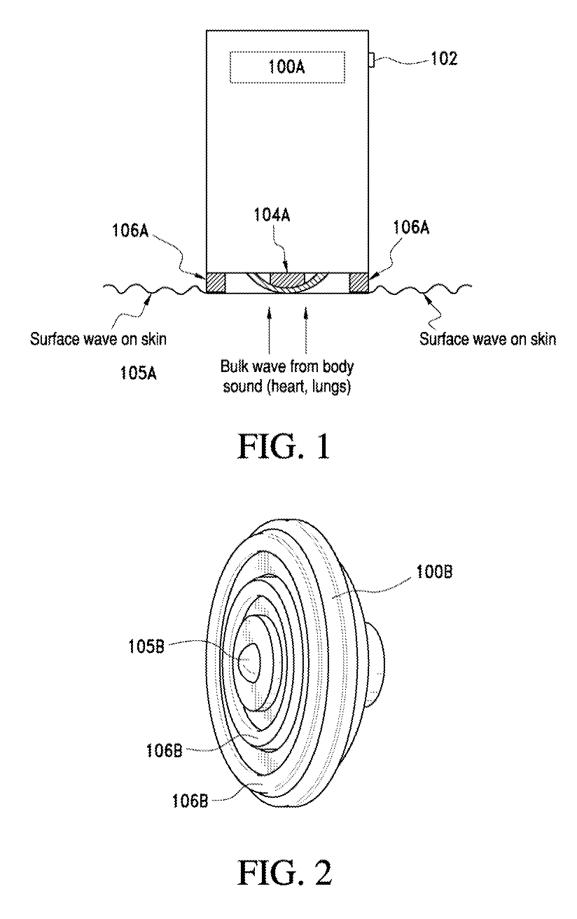

The invention preferably is a stethoscope with a suppression barrier present around the diaphragm/transducer. FIGS. 1-3 illustrate exemplary chest pieces for use with electronic stethoscopes.

FIG. 1 illustrates a partial cross-section sketch of an exemplary transducer housing portion (or chest piece) 100A of an electronic stethoscope. The electronics associated with the transducer itself are not shown. The exemplary sketch shows a connection point 102 to other components of the stethoscope and/or a port for connecting for example, to recording equipment, display equipment, or a PC connection. The exemplary sketch shows a transducer head 104A. A nipple 105A that covers the transducer head 104A is preferably removable and made in different sizes to accommodate different amounts of tissue compliance and feel any space between the transducer head 104A and the patient's skin. By having a removable nipple 105A, the transducer head 104A can be used as an ultrasound device (i.e., a Doppler device) with gel or other material providing a transmission medium between the transducer head 104A and the patient's skin.

The exemplary sketch shows a suppression barrier 106A extending from the housing 100A. The suppression barrier 106 is designed to contact the skin or body of the patient and acts to prevent SAW waves from entering the transducer region. The suppression barrier 106A may be made of a variety of materials. However, heavier materials tend to provide a better barrier for stopping waves which have both mass and energy. As illustrated, the transducer 104A and the suppression barrier 106A would be tangent to the same plane of air such that when the transducer housing is pressed against the patient, the transducer 104A and the suppression barrier 106A would both come in contact and touch the patient's tissue as illustrated in FIG. 1.

FIG. 2 illustrates an exemplary version of a transducer housing portion of an electronic stethoscope with a pair of O-rings 106B as the suppression barrier attached to the chest piece 100B. The O-rings 106B may reside within channels formed within the chest piece 100B or be simply attached to a flat surface on the chest piece 100B. When the O-rings 106B are simply attached to the chest piece, the O-rings may have a flat surface to better match the chest piece's flat surface. In the center of the face of the transducer housing 100B is the transducer nipple 105B, which is removable so that the transducer can be used in an ultrasound application.

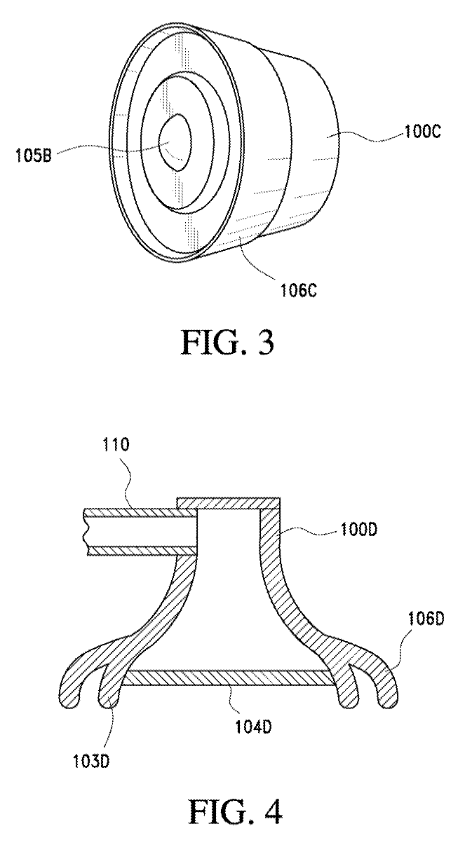

FIG. 3 illustrates an exemplary version of a transducer housing portion of an electronic stethoscope that includes multiple layers of tape 106C wrapped around the transducer housing and thus the transducer nipple 105C to provide the suppression barrier. The tape layers 106C extend approximately 0.25 inches beyond the transducer housing 100C. During testing in a 95-dBA simulated UH-60 noise environment, the user was able to hear and recognize a heartbeat of an individual, while without the tape layers 106C, the user was not able to hear the heartbeat of the individual.

An exemplary acoustical stethoscope is illustrated in FIG. 4. The illustrated stethoscope includes a suppression barrier 106D extending out from the chest piece 100D and down around the rim 103D and the diaphragm 104D as illustrated in cross-section in FIG. 4. FIG. 4 also illustrates tubing 110 connected to chest piece 100D. The illustrated suppression barrier 106D could be at least one O-ring.

Although the suppression barrier has been illustrated in FIGS. 2-4 as a pair of O-rings 106B, 106B, a jerry-rigged tape structure 106C and a member 106D, respectively, a variety of members could perform the suppression function such as any dampening member/component present around the transducer/diaphragm. Although what is illustrated are round suppression barriers, one of ordinary skill in the art will appreciate based on this disclosure that the suppression barriers may take a variety of shapes while still stopping SAW waves running along the patient's skin.

While the present invention has been described with respect to embodiments having one or two suppression barriers, multiple suppression barriers may be used. Also, the exemplary and alternative embodiments described above may be combined in a variety of ways with each other.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

The foregoing description describes different components of exemplary embodiments being “connected” to other components. These connections includes physical hardwired connections, wireless connections, magnetic connections, and other types of connections capable of carrying digital and/or analog information between the components.

Although the present invention has been described in terms of particular embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

I claim:

1. A stethoscope comprising:
a pair of ear pieces;
signal processing components connected to said ear pieces; and
a chest piece having
a housing,
a transducer connected to said signal processing components and said housing,
at least one suppression barrier attached to said housing.

2. The stethoscope according to claim 1, wherein said at least one suppression barrier extends from said housing.

3. The stethoscope according to claim 1, wherein said at least one suppression barrier surrounds said transducer.

4. The stethoscope according to claim 1, wherein said at least one suppression barrier includes two suppression barriers co-centered relative to said transducer.

5. The stethoscope according to claim 1, wherein said at least one suppression barrier includes at least one O-ring.

6. The stethoscope according to claim 1, wherein said at least one suppression barrier includes means for stopping surface waves from reaching said transducer.

7. The stethoscope according to claim 1, wherein said transducer includes a nipple,
said at least one suppression barrier and said transducer nipple are tangent to a plane running parallel to said surface of said chest piece such that when the chest piece is touched against a patient, each of these pieces is flush with the patient's tissue.

8. A chest piece for use in an electronic stethoscope comprising:
a housing;
signal processing components within said housing, said signal processing components includes a connection interface;
a transducer connected to said signal processing components; and
at least one suppression barrier attached to said housing and centered with respect to said transducer.

9. The chest piece according to claim 8, wherein said at least one suppression barrier extends from said housing.

10. The chest piece according to claim 8, wherein said at least one suppression barrier surrounds said transducer.

11. The chest piece according to claim 8, wherein said at least one suppression barrier includes two suppression barriers co-centered relative to said transducer.

12. The chest piece according to claim 8, wherein said at least one suppression barrier includes at least one O-ring.

13. The chest piece according to claim 8, wherein said at least one suppression barrier includes means for stopping surface waves from reaching said transducer.

14. The chest piece according to claim 8, wherein said transducer includes a nipple that is removable from said transducer,
said at least one suppression barrier and said transducer nipple are tangent to a plane running parallel to said surface of said chest piece such that when the chest piece is touched against a patient, each of these pieces is flush with the patient's tissue.

15. A stethoscope comprising:
ear pieces;
a binaural connected to said ear pieces;
tubing connected to said binaural;
a chest piece connected to said tubing, said chest piece having
a housing attached to said tubing,
a diaphragm or bell connected to said housing,
a rim around said diaphragm or bell; and
at least one suppression barrier attached to said housing and extending from said housing external to said rim.

16. The stethoscope according to claim 15, wherein said at least one suppression barrier surrounds said diaphragm or bell.

17. The stethoscope according to claim 15, wherein said at least one suppression barrier includes two suppression barriers co-centered relative to said diaphragm or bell.

18. The stethoscope according to claim 15, wherein said at least one suppression barrier includes at least one O-ring.

19. The stethoscope according to claim 15, wherein said at least one suppression barrier includes means for stopping surface waves from reaching said diaphragm or bell.

20. The stethoscope according to claim 15, wherein said transducer includes a nipple that is removable from said transducer,
said at least one suppression barrier and said transducer nipple are tangent to a plane running parallel to said surface of said chest piece such that when the chest piece is touched against a patient, each of these pieces is flush with the patient's tissue.
